(19) United States
(12) Patent Application Publication
(10) Pub. No.: US 2004/0106673 A1
(43) Pub. Date: Jun. 3, 2004

(54) Compositions and methods for inhibiting proliferation in human prostate cancer cells

(76) Inventor: Allan H. Conney, Princeton, NJ (US)

Correspondence Address: Jane Massey Licata, Licata & Tyrrell P.C., 66 E. Main Street, Marlton, NJ, 08053, US

(21) Appl. No.: 10/657,685

(22) Filed: Sep. 8, 2003

Related U.S. Application Data
(60) Provisional application No. 60/408,568, filed on Sep. 6, 2002.

(51) Int. Cl. A61K 31/337; A61K 31/22; A61K 31/203; A01N 31/04; A61K 31/015

(52) U.S. Cl. 514/449; 514/546; 514/559; 514/725; 514/763

(57) ABSTRACT

The present invention includes compositions and methods for treatment of prostate cancer which involve the use of 12-O-tetradecanoylphorbol-13-acetate combined with a retinoid such as all-trans-retinoic acid or paclitaxol, wherein the drugs are administered together, and further wherein the combined use of these agents results in a synergistic effect on prostate tumor cell growth.

### INTRODUCTION

This application claims the benefit of U.S. Provisional Application No. 60/408,568 filed Sep. 6, 2002.

### BACKGROUND OF THE INVENTION

12-O-tetradecanoylphorbol-13-acetate (TPA) and all-trans-retinoic acid (ATRA) belong to a class of compounds known to be inducers of cell differentiation. Such pharmacological activity of these individual compounds has been exploited in anti-cancer therapy where the goal is to inhibit growth of the cancerous cells. For example, TPA has been used to treat acute myelocytic leukemia in patients refractory to conventional therapies. Patients showed decreased numbers of myeloblasts in bone marrow and peripheral blood as well as temporary remission of disease symptoms without any reported serious toxicities (Han, Z. T. et al. 1998. Proc. Natl. Acad. Sci. USA 95:5357-5361). TPA has also been administered to patients enrolled in a Phase I clinical trial of patients with myeloid leukemia (Strair, R. K. et al. 2002. Clin. Cancer Res. 8:2512-2518). In an additional study, clinically achievable concentrations of TPA in combination with clinically achievable concentrations of ATRA, 1α,25-dihydroxyvitamin D3 or sodium butyrate synergistically inhibited the growth and stimulated the differentiation of cultured HL-60 myeloid leukemia cells, suggesting that a combination of these drugs may be more effective than TPA alone for the treatment of refractory myeloid leukemia patients (Zheng, X. et al. 2000. Oncol. Res. 12:419-427). In addition to effects in hematological cancers, TPA and ATRA have been studied individually in cells for potential effects in other types of cancer, including prostate cancer. Prostate cancer is the most common malignancy in men in the United States and the second most common cause of death in men over 55 years of age. Treatments for the disease vary depending on the stage of the disease when it is detected. Although surgery and radiation are common treatment modalities, chemotherapy is often employed. There is active research in the development of new and more effective chemotherapeutic agents.

Mechanistic studies with TPA and ATRA in prostate cancer cell lines have shown that both agents, when used singly, are capable of inducing apoptosis and inhibiting prostate tumor cell growth in vitro (e.g., De Coster, R. et al. 1992. J. Steroid Biochem. Molec. Biol. 43:197-201; Blagosklonny, M. V. et al. 1997. Cancer Res. 57:320-325; Garzotto, M. et al. 1998. Cancer Res. 58:2260-2264; Konno, S. Et al. 1996. Anticancer Res. 16:1843-1849). In the case of TPA, its effects in prostate cancer cells have demonstrated that androgen-dependent (e.g., LNCaP cells), but not androgen-independent (e.g., JCA-1 cells), cells lines are sensitive to TPA-induced growth inhibition (Powell, C. T. et al. 1996. Cell Growth Different. 7:419-428).

It has now been found that combination therapy where TPA is combined with drugs, such as ATRA and paclitaxol, produces synergistic effects in prostate cancer cells to inhibit tumor cell growth and thus treat prostate cancer.

### SUMMARY OF THE INVENTION

An object of the present invention is a composition for inhibition of prostate tumor cell growth and treatment of prostate cancer which comprises 12-O-tetradecanoylphorbol-13-acetate and a retinoid administered separately or together in pharmaceutically acceptable carriers or diluents.

Another object of the present invention is a method of inhibiting prostate tumor cell growth and treating prostate cancer which comprises contacting said cells with 12-O-tetradecanoylphorbol-13-acetate and a retinoid, administered separately or together, so that prostate tumor cell growth is inhibited.

Another object of the present invention is a composition for inhibition of prostate tumor cell growth and treatment of prostate cancer which comprises 12-O-tetradecanoylphorbol-13-acetate and paclitaxol administered separately or together in pharmaceutically acceptable carriers or diluents.

Another object of the present invention is a method of inhibiting prostate tumor cell growth and treating prostate cancer which comprises contacting said cells with 12-O-tetradecanoylphorbol-13-acetate and paclitaxol, administered separately or together, so that prostate tumor cell growth is inhibited.

### DESCRIPTION OF THE DRAWINGS

FIG. 1 depicts the effect of TPA treatment on LNCaP cell viability after incubation for 96 hours.

FIG. 2 depicts the synergistic effects of TPA and ATRA on the growth of LNCaP cells. Cells were treated with either TPA alone (0.32 nM), ATRA alone (0.5 to 10 μM), or a combination of the two agents.

FIG. 3 depicts the effects of treatment with vehicle (5 μl/g body weight), TPA (0.16 nmol/g; 5 μl vehicle/g body weight), ATRA (0.5 nmol/g; 5 μl vehicle/g body weight), or TPA in combination with ATRA (same doses), once a day for 21 days (13 mice/group), on LNCaP tumor cell growth in male NCr mice. Tumor cell growth was measured and expressed as percent of initial size.

FIG. 4 depicts rates of change in percent of initial tumor size between groups of mice treated with vehicle (control), TPA (0.16 nmol/g), ATRA ((0.5 nmol/g) or TPA+ATRA (same doses).

FIG. 5 depicts results from the second part of an in vivo study in male NCr mice (Group B animals), where mice were treated for an additional 25 days with vehicle (control), TPA (0.16 nmol/g), ATRA ((0.5 nmol/g) or TPA+ATRA (same doses). Results shown are the rate of tumor growth for the subsets of mice.

FIG. 6 depicts the tumor growth inhibitory effects in vivo in male Ncr mice of TPA combined in therapy with paclitaxol. Male NCr immunodeficient mice were injected subcutaneously with LNCaP cells. Treatments included vehicle, TPA (100 ng/g; 5 μl vehicle/g), paclitaxol (10 ng/g; 5 μl vehicle/g), and TPA (100 ng/g) in combination with paclitaxol (10 ng/g; 5 μl vehicle/g), once a day for 5 days followed by a 2 day intermission. The mice received treatment for 28 days.

FIG. 7 depicts the number of viable cells present after paclitaxol (4 or 10 ng/ml) treatment of LNCaP cells seeded at a density of 1×10⁵ cells/ml and incubated for 48 hours with either TPA (1 ng/ml) or vehicle.

### DETAILED DESCRIPTION OF THE INVENTION

The present invention includes compositions for treatment of prostate cancer that consists of a combination 30 of two drugs, both known to have activity separately to inhibit growth of prostate cancer cells in vitro. In each composition of the present invention one of the drugs is 12-O-tetradecanoylphorbol-13-acetate (TPA). This drug is then combined in therapy with either a retinoid, including but not limited to all-trans-retinoic acid (ATRA), or paclitaxol. These combinations of drugs have now been shown to have synergistic effects on prostate tumor cell growth. “Synergy” of pharmacological activity of drug products, defined as production of a pharmacological response with the combination of drugs that is greater than the effect of either drug alone, is not a commonly seen event and cannot be predicted based upon the known pharmacology of either agent individually. Although previous research had suggested that TPA combined with various other compounds (i.e., ATRA, sodium butyrate or 1α,25-dihydroxyvitamin D3) had a synergistic effect on growth and differentiation of cultured myeloid leukemia cells in vitro, suggesting clinical synergism between these agents, the synergistic effects of TPA with other chemotherapeutics has not been established in vivo nor in the treatment of solid tumors such as prostate tumors, nor in prostate cancer cell lines in vitro.

Therefore, studies were performed to assess the effects of various concentrations of TPA on the growth of prostate cancer cells (LNCaP cells) in vitro.

LNCaP cells were seeded in culture dishes and incubated for 24 hours to allow the cells to attach to the culture dishes. The cells were then treated with ethanol (2 μl/ml) or TPA in ethanol (0.2 to 10 ng/ml) for 96 hours.

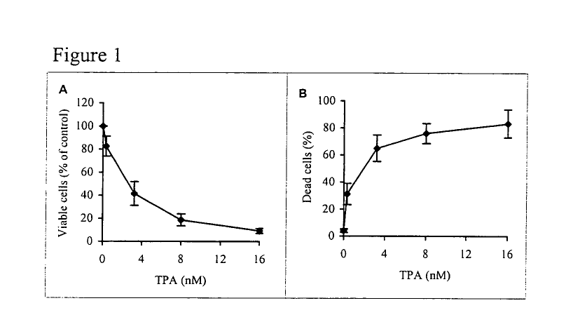

The number of viable and dead cells was determined by trypan blue exclusion. Treatment with TPA resulted in a dose-dependent decrease in the number of viable cells when compared to control cells treated only with ethanol (FIG. 1). The concentration of TPA that caused a 50% inhibition of LNCaP cell growth was 3 nM (IC₅₀). The IC₅₀ for TPA varied between 1 and 3 nM in different experiments. Treatment of these cells with 3.2 nM TPA resulted in morphologically distinct apoptotic cells. These data indicated that clinically achievable levels of TPA had a strong inhibitory effect on the proliferation and viability of LNCaP cells and that apoptosis was increased.

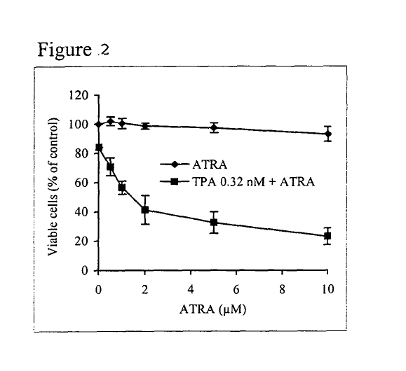

The potential synergistic effects of TPA and ATRA on the growth of LNCaP cells were then determined. Cells were treated with either TPA alone (0.32 nM), ATRA alone (0.5 to 10 μM), or a combination of the two agents (0.32 nM TPA and increasing concentrations of ATRA; FIG. 2). ATRA treatment alone had little effect on LNCaP cell growth, while TPA treatment alone had a slight inhibitory effect on cell growth. However, combination of ATRA with TPA resulted in a substantial increase in growth inhibition (FIG. 2). Analysis of the data by the method of Laska et al. (1994. Biometrics 50:834-841) revealed synergistic effects for the combination of TPA and ATRA. The synergistic effects were statistically significant at ATRA concentrations of 1, 2 and 5 μM (p<0.002, 0.001 and 0.001, respectively) and marginally significant at an ATRA concentration of 0.5 μM (p<0.068).

Although treatment of LNCaP cells with 0.16 or 0.32 nM TPA or with 1 μM ATRA alone for 96 hours had little or no effect on apoptosis, treatment of these cells with a combination of TPA and ATRA resulted in a marked stimulation of apoptosis as measured by the percent of pre-G₀/G₁ cells using flow cytometry. Untreated or ethanol-treated control cells incubated for 96 hours had 3-6.6% apoptotic cells and cells treated with TPA (0.16 or 0.32 nM) or ATRA (1 μM) had the same low level of apoptotic cells. In contrast to the lack of an apoptotic effect with these single drug treatments, treatment of LNCaP cells with 0.16 nM TPA and 1 μM ATRA for 96 hours resulted in 35% apoptotic cells and treatment of the cells with 0.32 nM TPA and 1 μM ATRA resulted in 62% apoptotic cells (Table 1).

TABLE 1
Effects of TPA Alone or in Combination with ATRA on Proliferation,
Cell Cycle Distribution an Apoptosis in Cultured LNCaP Cells

| | # Visible | | | | % |
|---|---|---|---|---|---|
| | Cells | % Cells | % Cells | % Cells | Apoptotic |
| Treatment | (1 × 10⁵) | in G₀/G₁ | in S | in G₂/M | Cells |
| Untreated | 10.9 | 70.9 | 16.6 | 8.7 | 3.5 |
| control | | | | | |
| Ethanol | 11.0 | 67.6 | 19.1 | 9.2 | 3.9 |
| TPA | 6.9 | 69.7 | 15.9 | 8.7 | 5.4 |
| (0.32 | | | | | |
| nM) | | | | | |
| ATRA | 11.1 | 73.3 | 14.8 | 8.2 | 3.5 |
| (1 μM) | | | | | |
| TPA (0.32 | 1.3 | 29.1 | 5.1 | 3.3 | 62.1 |
| nM) + | | | | | |
| ATRA (1 | | | | | |
| μM) | | | | | |

With the synergistic effects of TPA and ATRA in human prostate cancer cells established, experiments were performed in animals to establish that the effects would also be seen in vivo. Immunodeficient male NCr mice were used as they are a well-established mouse model for examination of human tumor cell growth. Intraperitoneal (i.p.) injection of 100 or 200 ng TPA per gram of body weight resulted in rapid systemic absorption of TPA, with peak blood levels occurring within 30-60 minutes. TPA was administered in a vehicle that consisted of propylene glycol, polysorbate 80, benzyl alcohol, ethanol, and water (40:0.5:1:10:48.5). Peak blood levels of TPA were in the range of 3 ng/ml (4.8 nM) after a 200 ng/g injection and 1 ng/ml (1.7 nM) after a 100 ng/g injection. The half-life of TPA in these mice was about 3-6 hours. No TPA was detectable in blood at 8, 12 or 24 hours following an i.p. injection of 100 ng/g TPA. Measurable blood levels of TPA were observed at 8 and 24 hours after the injection of 200 ng/g TPA. The i.p. injection of 100 ng/g TPA once a day for 3 weeks gave similar TPA blood level profiles as were observed after a single 100 ng/g i.p. injection of TPA. These data indicated that daily i.p. injections of TPA for 3 weeks did not inhibit or stimulate the metabolism of TPA, as well as indicating that there was good absorption of TPA following i.p. injection.

With the feasibility of the use of TPA by i.p. injection now established, male NCr mice were injected subcutaneously with LNCaP cells in matragel. Four weeks later, mice with well-established tumors (0.065 to 1 cm long and 0.65 to 1 cm wide; 4 mice/group) were injected i.p. with vehicle (5 μl/g body weight) or TPA (0.16 nmol/g; 5 μl vehicle/g body weight) once a day for the first two days. On day 3, the dose of TPA was increased (0.32 nmol/g; 5 μl vehicle/g body weight) and this dose level was administered i.p. for 11 days. The length and width of each tumor was measured at the start of the study and during treatment with TPA. Tumor size was expressed as cm² (length×width). There was a statistically significant inhibition of tumor cell growth in the TPA treated animals. The average tumor size in the vehicle-treated control group increased to about 200% of the initial tumor size while the average tumor size after 13 days of treatment with TPA was about 95% of the initial tumor size. The animals were sacrificed after 13 days of treatment because of excessive toxicity (decreased body weight and one death on day 14 of the study).

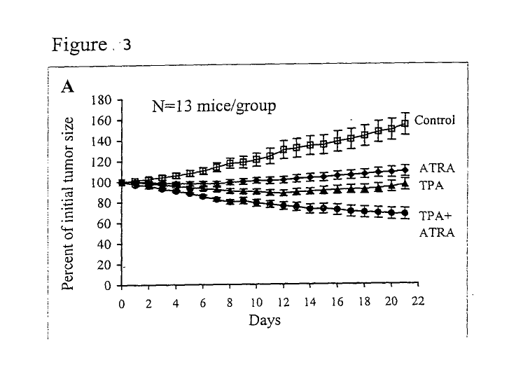

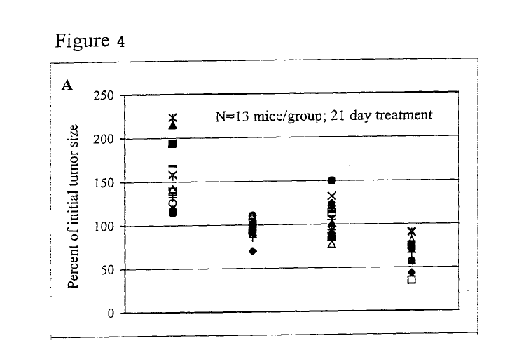

In a second study, male NCr mice with well-established LNCaP tumors were injected with vehicle (5 μl/g body weight), TPA (0.16 nmol/g; 5 μl vehicle/g body weight), ATRA (0.5 nmol/g; 5 μl vehicle/g body weight), or TPA in combination with ATRA (same doses), once a day for 21 days (13 mice/group). Tumor cell growth was measured and expressed as percent of initial size (FIG. 3). The rates of change in percent of initial tumor size were significantly different between the treatment groups and the control group, as well as between treatments. The percent of initial tumor size in each group was 154.2±10.2 (control), 96.4±3.1 (TPA), 109±5.5 (ATRA), and 67.6±4.8 (TPA plus ATRA) (FIG. 4). The effect of the various treatments on tumor growth or regression in individual mice is shown below in Table 2. In this table, mice in Group A (13 mice/group) were treated for 21 days, while mice in Group B (6 mice/group) were a subset of the original group and were treated for an additional 25 days.

TABLE 2
Effects of TPA, ATRA or Combined Treatment of TPA and
ATRA (i.p.) On Tumor Cell Growth or Regression in NCr Mice

| | | | | % of Mice | |
|---|---|---|---|---|---|
| | | | | with | % of Mice |
| | | | Duration | Tumor | with Tumor |
| Group | Treatment | # of Mice | (days) | Growth | Regression |
| A | vehicle | 13 | 21 | 100 | 0 |
| | ATRA | 13 | 21 | 69 | 31 |
| | TPA | 13 | 21 | 38 | 62 |
| | ATRA + | 13 | 21 | 0 | 100 |
| | TPA | | | | |
| B | vehicle | 6 | 46 | 100 | 0 |
| | ATRA | 6 | 46 | 83 | 17 |
| | TPA | 6 | 46 | 33 | 67 |
| | ATRA + | 6 | 46 | 17 | 83 |
| | TPA | | | | |

Results from the Group A animals showed that the combined treatment of TPA plus ATRA resulted in complete tumor growth inhibition and some tumor regression in all of the treated mice at 21 days. The effect of the 21 day treatments on body weight was examined in order to determine if there was the same level of toxicity seen in the first animal study. The results showed that there were no statistically significant effects on body weight in any of the treatment groups as compared to the vehicle control animals, except for the TPA+ATRA group. There was no significant relationship between changes in body weight and changes in tumor size in individual mice. These data demonstrated that there was no appreciable toxicity in the animals treated i.p. for 21 days with any of the tested regimens.

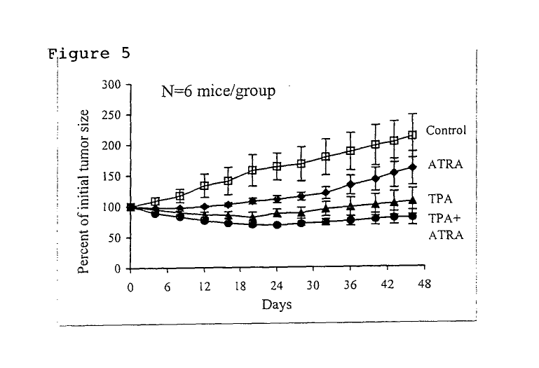

The results from the Group B animals were analyzed separately. Although the rate of tumor growth for the subset of 6 mice was similar to that of the entire group (13 mice/group; Group A data), at later time intervals, tumors in the ATRA group grew at the about the same rate as tumors in the vehicle control group (FIG. 5). The percent of initial tumor size for the entire 46 day treatment interval was: 211.1±35.2% (vehicle control); 106.1±21.0% (TPA); 159.2±27.0% (ATRA); and 78.2±11.3% (TPA+ATRA). Analysis of these data showed that there was a significant regression in the combined treatment group and the TPA only group as compared to control animals (Table 2). There were significant decreases in body weight in the animals treated for 46 days with either TPA alone or TPA combined with ATRA. The effects on body weight, however, did not appear to be related to changes in tumor growth and regression.

Considered together, these data showed that treatment of prostate tumor cells in vivo with TPA alone or in combination with ATRA resulted in a significant reduction in tumor cell growth and in tumor regression. The doses shown to be effective in vivo were without significant toxicity in vivo. These data are in agreement with other published data on TPA toxicity where doses of TPA in the range of 0.25 to 1 mg in humans produced only vein irritation and transient fever, and an occasional incidence of transient dyspnea (Han, Z. T. et al. 1998. Proc. Natl. Acad. Sci USA 95:5357-5361; Han, Z. T. et al. 1998. Proc. Natl. Acad. Sci USA 95:5362-5365; Strair, R. K. et al. 2002. Clin. Cancer Res. 8:2512-2518).

Further examination of the LNCaP tumors from the in vivo experiments revealed that there was a correlation between tumor size and tumor weight in individual mice r=0.932; CI=0.844-0.971). The percent of mitotic cells was decreased significantly in tumors from mice treated with TPA and ATRA but not in the other groups. Apoptosis as measured by the percent of caspase 3 (active form) positive cells in tumors was increased significantly in the TPA group and in the TPA+ATRA group, and the ratio of percent mitotic cells/percent caspase 3 positive cells was decreased in these groups (Table 3).

TABLE 3
Effects of TPA, ATRA, and Combined Treatments on Percent of Mitotic
Cells and Percent of Caspase 3 Positive Cells in LNCaP Tumors

| | | | | Ratio of |
|---|---|---|---|---|
| | | | % Caspase 3 | Mitotic |
| | | % Mitotic | Positive | Cells/Caspase |
| Treatment | # of Mice | Cells | Cells | 3 Cells |
| Control | 5 | 0.58 ± 0.06 | 0.31 ± 0.03 | 1.98 ± 0.31 |
| TPA | 6 | 0.50 ± 0.05 | 0.45 ± 0.03* | 1.13 ± 0.11* |
| ATRA | 6 | 0.54 ± 0.04 | 0.35 ± 0.03 | 1.62 ± 0.19 |
| TPA + | 6 | 0.39 ± 0.02* | 0.47 ± | 0.87 ± |
| ATRA | | | 0.04** | 0.11** |

*statistically significant as compared to control, p < 0.05
**statistically significant as compared to control, p < 0.01

There was a significant relationship in individual mice treated for 46 days between the percent of initial tumor size and the ratio of percent mitotic cells/percent caspase 3 positive cells r=0.853; CI=0.679-0.936).

These data demonstrate for the first time a synergistic effect of TPA and ATRA on prostate tumor cell growth both in vitro and in vivo. Although treatment of cells with low concentrations of TPA or ATRA alone had little to no effect on apoptosis, treatment with a combination of these agents resulted in significant levels of apoptosis (Table 1). Moreover, the concentrations needed to produce these effects (0.1 to 0.2 ng/ml or 0.16 to 0.32 nM for TPA and 1 μM ATRA) are levels are clinically achievable and relevant (Cui, X. X. et al. 2002. Oncol. Res. 13:169-174; Chen, G. Q. et al. 1996. Leukemia 10:825-826). Additionally, daily injections of ATRA and TPA inhibited growth of even well-established tumors in vivo (Table 2). The determination of blood levels of TPA indicated that clinically relevant doses were achieved in the mice and that information, combined with the known half-life of the drug, would be used by one of skill to design dosing regimens for patients with prostate tumors. Based on the data provided, it is possible that administration of lower doses of TPA administered two to three times daily would achieve more sustained blood levels and might be more effective than the once per day dosing regimen. One of skill would be able to determine the optimum dosing regimen based on the data provided herein.

The effects of TPA alone or in combination with ATRA on apoptosis in tumors was of importance, in particular the fact that the ratio of the percentage of mitotic cells to the percentage of apoptotic cells in the tumors from individual mice was closely associated with tumor growth during the 46 day treatment regimen. In an earlier study, Garzotto and colleagues (1999. Cancer Res. 59:5194-5201) demonstrated that gamma irradiation or an intravenous injection of TPA into the retro-orbital plexus had minimal effects on apoptosis in orthotopically transplanted LNCaP tumors in immunodeficient mice. However, treatment with a combination of gamma irradiation and TPA resulted in apoptosis in the tumors within 24 hours of treatment, as well as protective effects by TPA on radiation-induced damage to the colon. Therefore, the results of the present studies, considered together with the findings of Garzotti and colleagues suggest that clinical effects may be seen if TPA alone or in combination with ATRA is given in conjunction with gamma irradiation in patients with prostate cancer.

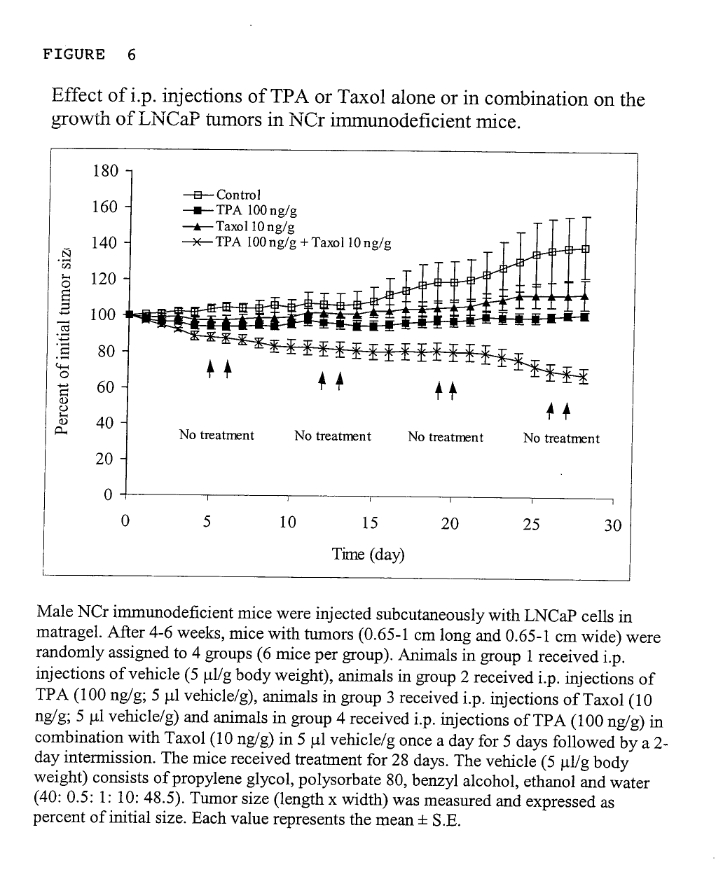

Studies were also performed to examine the potential synergistic activity of TPA combined in therapy with paclitaxol. Paclitaxol is a well-known chemotherapeutic agent sold under the trade name of Taxol® and currently approved by the U.S. Food and Drug Administration for the treatment of breast cancer, ovarian cancer, small cell lung carcinoma, and AIDS-related Karposi's sarcoma. Male NCr immunodeficient mice were injected subcutaneously with LNCaP cells in matragel. After 4 to 6 weeks, mice with tumors (0.65-1 cm long and 0.65-1 cm wide) were randomly assigned to 4 groups (6 mice per group). Animals in group 1 received i.p. injections of vehicle (5 μg body weight), animals in group 2 received i.p. injections of TPA (100 ng/g; 5 μl vehicle/g), animals in group 3 received i.p. injections of paclitaxol (10 ng/g; 5 μl vehicle/g), and animals in group 4 received i.p. injections of TPA (100 ng/g) in combination with paclitaxol (10 ng/g; 5 μl vehicle/g), once a day for 5 days followed by a 2 day intermission. The mice received treatment for 28 days. The vehicle consisted of polypropylene glycol, polysorbate 80, benzyl alcohol, ethanol and water (40:0.5:1:10:48.5, respectively). Tumor size was measured and expressed as percent of initial size. The results of these experiments are shown in FIG. 6. Combined treatment of TPA and paclitaxol (Taxol®) resulted in a synergistic effect. Tumor size was significantly smaller in animals treated with the combined therapy. Although treatment of LNCaP cells with TPA or paclitaxol alone for 48 or 96 hours had only small effects on apoptosis, treatment of these cells with a combination of TPA and paclitaxol, especially for 96 hours, resulted in a marked stimulation of apoptosis as measured by the percent of pre-G₀/G₁ cells using flow cytometry. These results are shown below in Table 4.

TABLE 4
Effects of TPA or Taxol Alone or in Combination on
Proliferation, Cell Cycle Distribution an Apoptosis in
Cultured LNCaP Cells

| | | | | | |
|---|---|---|---|---|---|
| | # Visible | | | | % |
| | Cells | % Cells | % Cells | % Cells | Apoptotic |
| Treatment | (1 × 10⁵) | in G₀/G₁ | in S | in G₂/M | Cells |

Treatment for 48 Hours

| | | | | | |
|---|---|---|---|---|---|
| Untreated | 8.9 | 76.5 | 16.1 | 5.2 | 1.6 |
| control | | | | | |
| Ethanol | 0.0 | 76.8 | 16.4 | 4.6 | 1.8 |
| TPA | 3.7 | 77.8 | 13.0 | 5.5 | 10.1 |
| 1 ng/ml | | | | | |
| Taxol | 4.6 | 73.0 | 12.8 | 6.4 | 12.9 |
| 5 ng/ml | | | | | |
| TPA + | 1.3 | 58.6 | 12.6 | 5.3 | 28.3 |
| Taxol | | | | | |
| (doses as | | | | | |
| above) | | | | | |

Treatment for 96 Hours

| | | | | | |
|---|---|---|---|---|---|
| Untreated | 10.6 | 85.7 | 7.5 | 5.2 | 1.2 |
| control | | | | | |
| Ethanol | 12.3 | 83.6 | 7.9 | 6.2 | 1.9 |
| TPA | 3.2 | 66.7 | 12.3 | 9.5 | 11.2 |
| 1 ng/ml | | | | | |
| Taxol | 2.5 | 49.7 | 12.4 | 8.7 | 28.4 |
| 5 ng/ml | | | | | |
| TPA + | 0.4 | 29.9 | 10.1 | 8.0 | 50.8 |
| Taxol | | | | | |
| (doses as | | | | | |
| above) | | | | | |

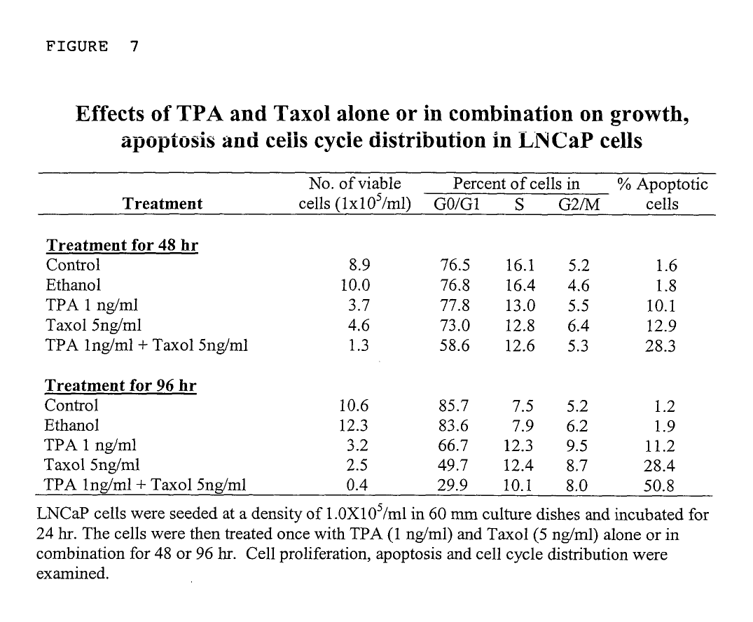

Experiments were also performed to determine the number of viable cells present after paclitaxol (4 or 10 ng/ml) treatment of LNCaP cells seeded at a density of 1×10⁵ cells/ml and incubated for 48 hours with either TPA (1 ng/ml) or vehicle. Treatment of cells in culture with paclitaxol in combination with TPA resulted in a synergistic effect on cell viability (see FIG. 7). Therefore, a combination of TPA and paclitaxol also was shown to be an effective treatment for inhibition of prostate tumor cell growth.

The present invention is therefore a composition for the treatment of prostate cancer in patients that comprises a combination therapy of an effective dose of TPA and an effective dose of a retinoid, such as ATRA, or paclitaxol, given in separate formulations (pharmaceutically acceptable formulations). In the context of the present invention an effective dose is one that produces an inhibition of tumor size and tumor cell growth, leading to tumor regression. One of skill would understand how to choose effective doses for TPA, the retinoid, or paclitaxol based on the results of the in vitro and in vivo studies presented herein, as well as what is known concerning the toxicity and efficacy of these agents for other uses. The doses would be ones that produce significant clinical effects with little toxicity. The pharmaceutical vehicle chosen for use would be ones routinely used in the art and would include but not be limited to saline, as well as intravenous formulations listed in U.S. Pat. No. 6,063,814. The TPA and retinoid or paclitaxol treatment would be administered to the patient by any route that one of skill would choose as being clinically relevant, including intravenous, intraperitoneal, or oral, where routes of administration can be different for the two drugs combined in the therapy. As discussed above, the combined TPA/retinoid or TPA/paclitaxol therapy may also be used in conjunction with other cancer treatments in order to maximize the efficacy of the chosen treatment regimen. The retinoids that might be used in this invention would include but not be limited to ATRA or derivatives of ATRA. Also contemplated as part of this invention for use in combination with TPA would be other differentiating agents that would include but not be limited to vitamin D and its derivatives and butyrate.

The present invention is also a method for inhibiting prostate tumor cell growth in a patient having a prostate tumor that involves administration of an effective dose of the TPA/retinoid or TPA/paclitaxol combination therapy so that tumor cell growth is inhibited. The present invention is thus also a method of treating prostate cancer in a patient that involves administration of an effective dose of the TPA/retinoid or TPA/paclitaxol combination therapy so that the prostate tumor regresses and the cancer is treated.

The following non-limiting examples are provided to further illustrate the claimed invention.

### EXAMPLES

### Example 1

### Cell Culture

LNCaP cells were obtained from the American Type Culture Collection (Rockville, Md., USA). TPA was obtained from Alexis Co. (San Diego, Calif.). ATRA was obtained from Sigma Chemical Co. (St. Louis, Mo.). LNCaP cells were maintained in RPMI-1640 culture medium containing 10% fetal bovine serum that was supplemented with penicillin (100 units/ml), streptomycin (100 μg/ml) and L-glutamine (300 μg/ml) as described (Zheng, X. et al. 2000. Oncol. Res. 12:419=427). Cultured cells were grown at 37° C. in a humidified atmosphere of 5% CO₂ and were passed twice a week. In all experiments, LNCaP cells were initially seeded at a density of 1×10⁵ cells/ml in a final volume of 2 or 5 ml in 35 or 60 mm tissue culture dishes, respectively.

### Example 2

### Determination of the Number of Viable Cells

To quantify the number of viable cells after each treatment, cells were counted using a hemacytometer under a light microscope. Cell viability was determined by the trypan blue exclusion assay, which was done by mixing 80 μl of cell suspension and 20 μl of 0.4% trypan blue solution for 2 minutes. Blue cells were counted as dead cells and the cells that did not absorb dye were counted as live cells.

### Example 3

### Flow Cytometry

To perform the analysis, 1×10⁶ cells were washed with phosphate-buffered saline (PBS) and resuspended in 500 μl of stain solution (20 mg/ml polyethylene glycol 8000, 50 μg/ml propidium iodide, 0.1% Triton-X-100, and 0.4 M sodium chloride). The mixture was incubated at 4° C. overnight in the dark before being analyzed on a Coulter Epics-Profile II flow cytometer. Propidium iodide is a fluorescent dye that intercalates into the DNA double helix. Whether cells are in the G₁ phase where they have two copies of their genome or in the G₂/M phase where they have 4 copies of their genome, or in the S phase where their DNA is between the two states, can be determined by the amount of propidium iodide that intercalates into the DNA. Pre G₀/G₁ cells were considered apoptotic cells. The proportion of cells in each phase of the cell cycle was calculated using the cytologic software provided.

### Example 4

### Treatment of NCr Mice

Male Ncr immunodeficient mice (6 to 7 weeks old) were obtained from Taco Farms (Germantown, N.Y.). The animals were housed in sterile filter-capped microisolator cages and provided with sterilized food and water. Prostate cancer cells (2×10⁶ cells/0.1 ml/mouse) suspended in 50% matrigel (Collaborative Research, Bedford, Mass.) in RPMI 1640 medium were injected subcutaneously into the right flank of the mice. After 4 to 6 weeks, mice with well-established tumors (0.65 to 1 cm by 0.65 to 1 cm) were used for the various experiments. In all experiments, animals received the same volume of injections (5 μl/g body weight). Tumor size and body weight were measured daily. At the end of a study, mice were sacrificed by decapitation, tumors were excised, weighed, and placed in phosphate-buffered formalin for 48 hr at room temperature and then placed in ethanol for 48 hr before preparing paraffin sections.

### Example 5

### Blood Levels of TPA

The concentration of TPA in blood after an i.p. injection of TPA was measured by a bioassay that quantifies ethyl acetate-extractable differentiating activity in blood (Cui, X. X. et al. 2002. Oncol. Res. 13:169-174). Differentiating activity was measured in HL-60 cells. Anticipated de-esterified metabolites of TPA do not interfere with this assay since they are not active in the bioassay. Although blood levels of bioactive material are expressed as ng of TPA/ml blood, the assay could include biologically active metabolites.

### Example 6

### Caspase 3 (Active form) Immunostaining

Affinity-purified polyclonal rabbit antibody that reacts with the p17 subunit of human and mouse caspase 3 but does not react with the precursor form was purchased (R&D Systems). Tumor sections used were stained by the horseradish peroxidase-conjugated-avidin method. Endogenous peroxidase was blocked by incubating the tissue sections in 3% hydrogen peroxide in methanol for 30 minutes at room temperature. Sections were then treated with 0.01 M sodium citrate buffer (pH 6.0) in a microwave oven at high temperature for 10 minutes. The sections were incubated with a protein block (normal goat serum) for 10 minutes, followed by avidin D for 15 minutes and biotin blocking solution for 15 minutes at room temperature. The sections were incubated with caspase 3 primary antibody (1:2000 dilution) for 30 minutes at room temperature followed by incubation with a biotinylated anti-rabbit secondary antibody for 30 minutes and incubation with conjugated-avidin solution for 30 minutes. Color development was achieved by incubation with 0.02% 3,3′-diaminobenzidine tetrahydrochloride containing 0.02% hydrogen peroxide for 10 minutes at room temperature. The slides were then counterstained with hematoxylin, dehydrated and coverslips were added for permanent mounting. A positive reaction was shown as a light brown to dark brown precipitate in the cytoplasm and/or perinuclei of the cells.

### Example 7

### Clinical Effects of Combined TPA/ATRA Therapy in Prostate Cancer Patients

A clinical study with a combination of TPA and ATRA will be performed using intravenously administered TPA (0.125-0.500 mg/m²), three to seven days per week, and ATRA (5-15 mg/m²), three to seven days per week, to achieve a blood level of 0.25-1 μM ATRA. Patients will be monitored for their response to these combined agents by monitoring tumor size.

What is claimed:

1. A composition for inhibition of prostate tumor cell growth comprising 12-O-tetradecanoylphorbol-13-acetate and a retinoid administered together or separately in pharmaceutically acceptable carriers or diluents.

2. A composition for treatment of prostate cancer in a human comprising 12-O-tetradecanoylphorbol-13-acetate and a retinoid administered together or separately in pharmaceutically acceptable carriers or diluents.

3. A method of inhibiting prostate tumor cell growth comprising contacting said cells with the composition of claim 1 so that prostate tumor cell growth is inhibited.

4. A method of treating prostate cancer in human comprising administering to said human a therapeutically effective amount of the composition of claim 1.

5. A composition for inhibition of prostate tumor cell growth comprising 12-O-tetradecanoylphorbol-13-acetate and paclitaxol administered together or separately in pharmaceutically acceptable carriers or diluents.

6. A composition for treatment of prostate cancer in a human comprising 12-O-tetradecanoylphorbol-13-acetate and paclitaxol administered together or separately in pharmaceutically acceptable carriers or diluents.

7. A method of inhibiting prostate tumor cell growth comprising contacting said cells with the composition of claim 5 so that prostate tumor cell growth is inhibited.

8. A method of treating prostate cancer in human comprising administering to said human a therapeutically effective amount of the composition of claim 5.
